(19) United States
(12) Patent Application Publication
(10) Pub. No.: US 2026/0174440 A1
(43) Pub. Date: Jun. 25, 2026

(54) ASPIRATION OR OCCLUSION TO PREVENT PROXIMAL MIGRATION OF INJECTED EMBOLIC SOLUTION DURING ENDOVASCULAR EMBOLIZATION TREATMENT IN A VESSEL

(71) Applicant: Neuravi Limited, Galway (IE)

(72) Inventors: David QUINN, Galway (IE); Karl KEATING, Galway (IE); Gillian GUNNING, Galway (IE); Ray McCARTHY, Galway (IE); Soraya SALINAS FERNANDEZ, Galway (IE); Michael SCANLON, Galway (IE)

(21) Appl. No.: 19/397,271

(22) Filed: Nov. 21, 2025

Related U.S. Application Data
(60) Provisional application No. 63/738,660, filed on Dec. 24, 2024.
(60) Provisional application No. 63/738,669, filed on Dec. 24, 2024.

(51) Int. Cl. A61B 17/12; A61B 17/00; A61M 25/00

(52) U.S. Cl. CPC A61B 17/12186; A61B 17/12031; A61B 17/12109; A61M 25/0029; A61B 2017/00849; A61B 2017/00942; A61B 2017/1205; A61B 2017/12127; A61B 2217/005; A61M 2025/0042

(57) ABSTRACT

Method for embolization treatment at a target site within a vessel using an endovascular embolization system. A microcatheter is navigated to the target site in the vessel. Via the microcatheter, only embolic solution is injected into the vessel. Aspirated fluid representing only a portion of the injected embolic solution present in the vessel is aspirated into the microcatheter to prevent the injected embolic solution present in the vessel from proximally migrating in the vessel. Alternatively, the injected embolic solution is prevented from proximal migration by occluding the vessel with the distal end of the microcatheter.

### CROSS REFERENCE TO RELATED APPLICATIONS

This application claims the benefit of priority under 35 U.S.C. § 119 to prior filed U.S. Provisional Patent Application No. 63/738,660 , filed Dec. 24, 2024 (Attorney Docket No.: 243382.000591(NRV6149USPSP2)), the entire contents of which is hereby incorporated by reference in its entirety as if set forth in full herein. This application also claims the benefit of priority under 35 U.S.C. § 119 to prior filed U.S. Provisional Application No. 63/738,669 , filed Dec. 24, 2024 (Attorney Docket No.: 243382.000592 (NRV6149USPSP3)), the entire contents of which is hereby incorporated by reference in its entirety as if set forth in full herein.

### FIELD

The present disclosure generally relates to an endovascular embolization treatment by injecting a liquid embolic agent (e.g., liquid glue material) into the vasculature occluding or blocking the supply of blood flow to a target site (e.g., hematoma) experiencing subdural bleeding. By way of example, the target site for the endovascular embolization treatment may be the middle meningeal artery (MMA). In particular, the present disclosure is directed to an improved endovascular embolization treatment that uses aspiration or occlusion to prevent undesirable proximal migration resulting from back pressure build-up of the injected liquid embolic solution.

### BACKGROUND

Endovascular treatment is widely performed to occlude or block blood supply (e.g., embolization) at a target site in a vessel. Embolization treatment may occur anywhere in the body, for example, in the middle meningeal artery (MMA). During endovascular embolization treatment a liquid embolic solution (e.g., solution of a liquid embolic agent (e.g., n-butyl-cyanoacrylate (n-BCA) or other liquid glue material) and an oil) may be injected into the vessel at the target site (e.g., subdural hematoma). As a result of back pressure build-up, the injected embolic solution undesirably migrates in a proximal direction resulting in one or more problems: (i) potential risk of unintentional occlusion of vessels at a location proximally of the target site; (ii) clogging of the lumen of the microcatheter with the injected embolic solution preventing tracking over a guidewire received therein; (iii) and/or adherence of the injected embolic solution to the exterior surface (i.e., outer wall) of the microcatheter.

It is therefore desirable to develop an improved endovascular embolization treatment that prevents or minimizes risk of proximal migration of the injected embolic solution while also preventing adherence of the embolic solution in the lumen of the microcatheter and to the exterior surface of the microcatheter.

### SUMMARY

An aspect of the present disclosure relates to an improved endovascular embolization treatment preventing or minimizing risk of proximal migration of the injected embolic solution by applying aspiration.

Another aspect of the present disclosure relates to an improved endovascular embolization treatment preventing adherence to the exterior surface of the microcatheter of the injected embolic solution by applying aspiration.

While yet another aspect of the present disclosure is directed to an improved endovascular embolization treatment that prevents unintended occluding of vessel(s) proximally of the target site by applying aspiration.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A is side view of an example endovascular embolization system with a dual lumen microcatheter in accordance with the present disclosure navigated to a target site in a vessel; wherein in the longitudinal/axial direction the inlet port of the aspiration lumen for receiving the aspirated fluid is aligned with the outlet port of the embolic solution lumen;

FIG. 1B is a longitudinal cross-sectional view of an example concentrically arranged dual lumen microcatheter in the endovascular embolization system in FIG. 1A;

FIG. 1C is a radial cross-sectional view along lines 1C-1C of FIG. 1B;

FIG. 2A is side view of another example endovascular embolization system with an eccentrically arranged dual lumen microcatheter in accordance with the present disclosure navigated to a target site in a vessel; wherein in the longitudinal/axial direction the outlet port of the embolic solution lumen and the inlet port of the aspiration lumen for receiving the aspirated fluid are substantially aligned with one another coinciding with the distal end/tip of the microcatheter;

FIG. 2B is a longitudinal cross-sectional view of the eccentric arrangement of the dual lumen microcatheter in the endovascular embolization system in FIG. 2A;

FIG. 2C is a radial cross-sectional view along lines 2C-2C of FIG. 2B;

FIG. 3 is an example hub connectable to the proximal end of the microcatheter (single lumen or dual lumen); wherein the hub is includes two lure connectors; one luer connector fitted with an ampule sealed via a removable locking tab, the sealed ampule containing preloaded and premixed embolic solution (e.g., embolic agent and oil); the other luer connector fitted to a vacuum source (e.g., vacuum pump);

FIG. 4 is a flow chart of the method of operation of the endovascular embolization system in accordance with the present disclosure in which aspiration is used to prevent proximal migration of the injected embolic solution.

FIG. 5A is side view of an example endovascular embolization system wherein the vessel is occluded preventing proximal migration of subsequently injected embolic solution via a self-actuating radially expandable occluding member disposed at a distal end of the microcatheter; the self-actuating radially expandable occluding member is illustrated in a radially constricted state;

FIG. 5B is a side view of the example endovascular embolization system of FIG. 5A with the self-actuating radially expandable occluding member illustrated in the radially enlarged (i.e., expanded) state;

FIG. 5C is a radial cross-sectional view along lines 5C-5C of FIG. 5A;

FIG. 5D is a radial cross-sectional view along lines 5D-5D of FIG. 5A;

FIG. 6 is a side view of another example endovascular embolization system wherein the vessel is occluded preventing proximal migration of subsequently injected embolic solution using a sail to foster distal advancement of a uniform non-radially expanding distal end/tip of the microcatheter to a wedged position in the vessel;

FIG. 7A is a side view of still another example endovascular embolization system wherein the vessel is occluded preventing proximal migration of subsequently injected embolic solution by creating a lower friction coefficient distal section of the microcatheter relative to a higher friction coefficient main shaft section proximally thereof fostering distal advancement of a uniform non-radially expanding distal end/tip of the microcatheter to a wedged position in the vessel; wherein the outer diameter of the distal section and the outer diameter of the main shaft section of the microcatheter are substantially equal;

FIG. 7B is a radial cross-sectional view through the distal section of the microcatheter along lines 7B-7B of FIG. 7A;

FIG. 7C is a radial cross-sectional view through the main shaft section of the microcatheter along lines 7C-7C of FIG. 7A;

FIG. 7D is a side view of yet another example endovascular embolization system wherein the vessel is occluded preventing proximal migration of subsequently injected embolic solution by creating a lower friction coefficient distal section of the microcatheter relative to a higher friction coefficient main shaft section proximally thereof fostering distal advancement of a uniform non-radially expanding distal end/tip of the microcatheter to a wedged position in the vessel; wherein the outer diameter of the distal section is larger than the outer diameter of the main shaft section of the microcatheter; and the outer diameter of the distal section of the microcatheter is larger than the inner diameter of the vessel through which it is being advanced;

FIG. 7E is a radial cross-sectional view through the distal section of the microcatheter along lines 7E-7E of FIG. 3D;

FIG. 7F is a side view of still another example endovascular embolization system wherein the vessel is occluded preventing proximal migration of subsequently injected embolic solution by creating a lower friction coefficient distal section of the microcatheter relative to a higher friction coefficient main shaft section proximally thereof fostering distal advancement of a uniform non-radially expanding distal end/tip of the microcatheter to a wedged position in the vessel; wherein the outer diameter of the distal section is larger than the outer diameter of the main shaft section of the microcatheter; and the outer diameter of the distal section of the microcatheter is substantially equal to the inner diameter of the vessel through which it is being advanced;

FIG. 8 is a side view of yet another example endovascular embolization system wherein the vessel is occluded preventing proximal migration of subsequently injected embolic solution by applying ultrasonic vibrations to a distal end/tip of the microcatheter fostering distal advancement to a wedged position in the vessel;

FIG. 9 is an example hub connectable to the proximal end of the microcatheter (single lumen or dual lumen); wherein the hub includes a lure connector fitted with an ampule sealed via a removable locking tab, the sealed ampule containing preloaded and premixed embolic solution (e.g., embolic agent and oil); and

FIG. 10 is a flow chart of the method of operation of the endovascular embolization system in accordance with the present disclosure in which occlusion of the vessel via the distal end/tip of the microcatheter itself prevents proximal migration of the subsequently injected embolic solution.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the term “microcatheter” is a catheter having a diameter that is small in comparison to catheters in cardiovascular applications, i.e. 8 French or less.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

Various example embolization treatment systems described and illustrated herein in accordance with the present disclosure stop bleeding at a desired target site (e.g., in the Middle Meningeal Artery (MMA)) by injecting into the vessel an embolic solution (e.g., embolic agent such as n-butyl-cyanoacrylate (n-BCA) and oil in any desired ratio)). Despite the advantages associated with such endovascular treatment, one significant drawback is back pressure from build-up of the injected embolic solution resulting in undesirable migration of the injected embolic solution within the vessel in a proximal direction relative to the target site in which it was administered via the outlet port of the microcatheter. Several concerns arise from the proximal migration in the vessel of the injected embolic solution. Vessel(s) downstream in the vasculature relative to the target site of the injected embolic solution may unintentionally and undesirably become occluded from proximal migration of the injected embolic solution. In addition, the injected embolic solution that migrates proximally adheres to the exterior surface of the microcatheter hampering or preventing withdraw from the body. Still further the injected embolic solution may clog the lumen of the microcatheter prohibiting tracking of the guidewire therein. The issue of proximal migration of the injected embolic solution in the vessel is addressed by the present endovascular embolization system and method of treatment by the simultaneous (i.e., in tandem or at the same time) or non-simultaneous (i.e., sequential, independent or not at the same time) aspiration from the vessel of fluid including blood and/or excess injected embolic solution. In addition to overcoming the issue of proximal migration of the injected embolic solution, in certain configurations or examples illustrated herein and described below, the aspirated fluid creates a suction distally of the target site of injection of embolic solution glucose solution assisting in pushing the injected embolic solution further distally into the vessel beyond the limited reach of the microcatheter.

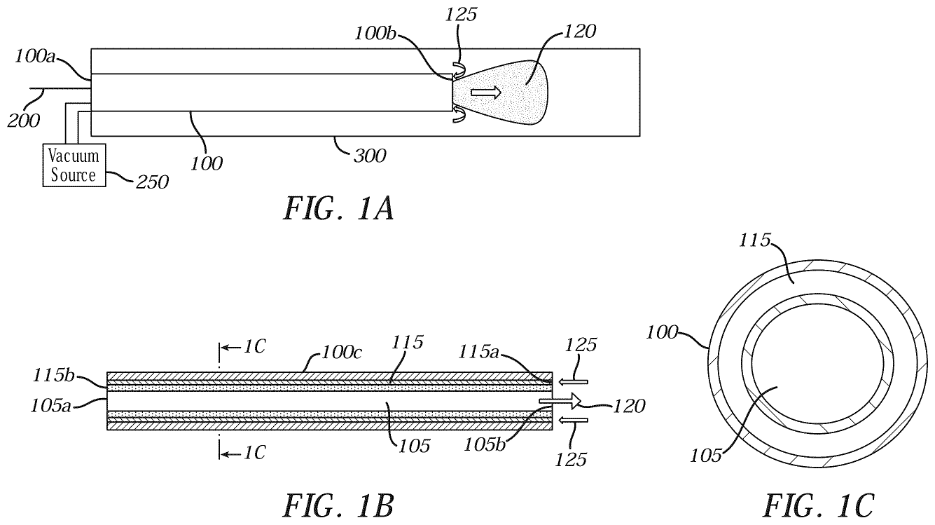

Several illustrative example configurations of an endovascular embolization system delivering the injected embolic solution into the vessel using a multi-lumen microcatheter 100 are disclosed herein (e.g., a dual lumen microcatheter 100 having two lumen 105, 115 separate and independent of one another). For example, microcatheter 100 depicted in FIGS. 1A-1C includes an embolic solution lumen 105 that tracks over a guidewire 200 receivable therein during navigation of the microcatheter 100 to the target site in the vessel 300 and subsequent delivery of the embolic solution 120. Fluid 12 (e.g., blood and/or excess injected embolic solution) from the vessel is aspirated is into an aspiration lumen 115 separate from the embolic solution lumen 105. More than two lumens are contemplated, for example, an optional third lumen (e.g., dedicated guidewire lumen) separate and independent from each the embolic solution lumen 105 and the aspiration lumen 115.

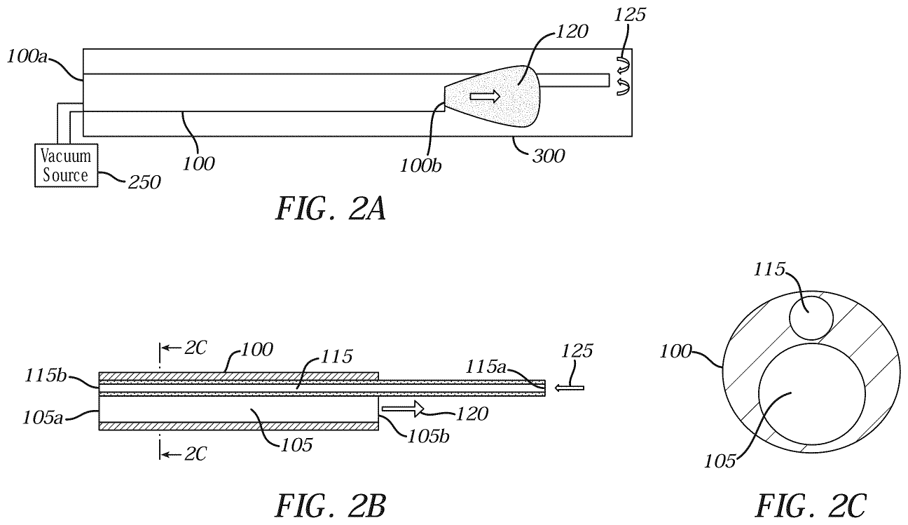

Arranged concentrically radially outward of the embolic solution lumen 105, the aspiration lumen 115 has an inlet port 115a at the distal end 100b of the microcatheter for receiving aspirated fluid (e.g., blood and/or excess injected embolic solution 120) and an outlet port 115b at an opposite proximal end 100a of the microcatheter 100. An outlet port 105b of the embolic solution lumen 105 coincides with the distal end 100b of the microcatheter 100, while the inlet port 105a of the embolic solution lumen 105 is aligned with the proximal end 100a of the microcatheter 100. A vacuum pressure source xx applies a negative or vacuum pressure to the proximal end 115a of the aspiration lumen 115 generating a suction or vacuum drawing (i.e., aspirating) aspirated fluid (e.g., blood and/or excess injected embolic solution 120) into the aspiration lumen 115 via the inlet port 115a. As illustrated in the side view of FIG. 1A, the embolic solution 120 is injected via the outlet port 105b of the embolic solution lumen 105 at the target site of the bleeding to be treated in the vessel 300. Vacuum or negative pressure created draws (i.e., aspirates) excess embolic solution 120 into the aspiration lumen 115 via the inlet port 115a counterbalancing back pressure build-up thereby preventing proximal migration of the injected embolic solution 120. In addition, the aspiration of excess embolic solution 120 into the aspiration lumen 115 via the inlet port 115a prevents adherence of the injected embolic solution 120 to the exterior surface of the microcatheter 100. Still further, the aspirated excess embolic solution 120 into the aspiration lumen 115 prevents unintended occlusion of a vessel proximally of the target site. Different arrangements of the respective embolic solution lumen 105 and aspiration lumen 115 are possible for the microcatheter in FIG. 1A. For instance, the embolic solution lumen 105 and aspiration lumen 115 may be arranged eccentrically of one another (similar to that shown in the radial cross-sectional view of FIG. 2C) while the inlet port 115a of the aspiration lumen 115 and the outlet port 105b of the embolic solution lumen aligned with one another at the distal end/tip 100b of the microcatheter 100.

Regardless of the arrangement (e.g., concentric or eccentric), the size, shape and arrangement of each of the lumens 105, 115 may be selected, as desired. Injection of the embolic solution 120 and application of suction (i.e., aspiration or negative pressure) may occur either simultaneously (i.e., in tandem or at the same time) or non-simultaneously (i.e., sequentially, independently or not at the same time). In the case of applying suction non-simultaneously with injection of the embolic solution 120, the order, timing, duration and volume dispensed of each of these operations may be selected, as desired.

In a next example in FIGS. 2A-2C, the embolic solution lumen 105 and the aspiration lumen 115 are arranged eccentrically of one another with an outlet port 105b of the embolic solution lumen 105 disposed proximally relative to the inlet port 115a of the aspiration lumen 115 via which blood is drawn or aspirated. Vacuum or negative pressure in the aspiration lumen 115 upon reaching the inlet port 115a at the distal end 100b of the microcatheter 100 produces a suction (i.e., a negative, lower or reduced pressure region) in the vessel 300 distally relative to the outlet port 105b of the embolic solution lumen 105. This generated region of negative pressure in the vessel 300 assists in pulling or drawing the more proximal injected embolic solution 120 further in the distal direction into the vessel 300 towards the target site to be treated. Hence, the push created by the injection of the embolic solution 120 in combination with the pull (i.e., draw or suction) from the region of negative pressure created in the vessel 300 by the vacuum source 250 ensures that the injected embolic solution 120 reaches the target site in the vessel 300. Moreover, the drawing distally of the injected embolic solution 120 counterbalances back pressure produced by the injected embolic solution thereby preventing proximal migration of the injected embolic solution. Still further, the drawing in the distal direction of the injected embolic solution 120 created by the negative pressure region prevents adherence to the exterior surface of the microcatheter 100 of the injected embolic solution 120. Moreover, the suction or drawing distally in the vessel 300 of the injected embolic solution 120 prevents unintended occlusion of vessels proximally of the target site. Arrangement of the respective embolic solution lumen 105 and aspiration lumen 115 in FIGS. 2A-2C are eccentric of one another but could alternatively have a concentric arrangement (similar to that shown in the radial cross-sectional view of FIG. 1C). In such alternative arrangement, the outer concentric aspiration lumen 115 is disposed radially outward of the central (inner) concentric embolic solution lumen 105 with the inlet port 115a of the aspiration lumen 115 being disposed in the longitudinal/axial direction distally of the outlet port 105a of the embolic solution lumen 105.

In any of the examples described above, aspiration may occur simultaneously (i.e., in tandem or at the same time) or non-simultaneously (i.e., independently, sequentially or not at the same time) as injection of the embolic solution 120.

A hub or syringe barrel may be attached to the proximal end of any of the microcatheter configurations illustrated and described herein to deliver the embolic solution 120 while subject to aspiration either simultaneously (i.e., in tandem or at the same time) or non-simultaneously (i.e., independently, sequentially or not at the same time).

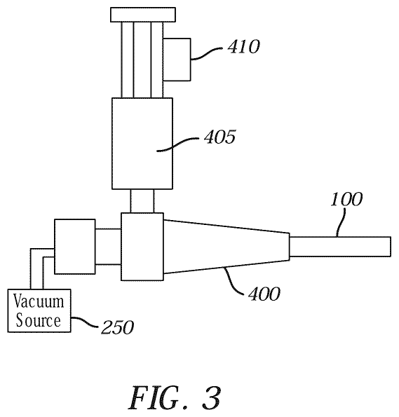

FIG. 3 is an example dual channel hub 400 in which an ampule 405 preloaded with a predefined volume of the embolic solution 120 (e.g., premixture in a desired ratio of an embolic agent and oil) is attached or fitted onto a first lure connector of hub 400. Ampule 405 is sealed thereby preventing premature dispensing of the contents stored therein. For example, ampule 405 may include a corresponding locking tab 410 that when removed, disrupted or broken by the physician or interventionalist dispenses the preloaded premixed solution stored therein. A vacuum source 250 is attached to hub 400 via a second lure connector. In response to removing, disrupting or breaking a seal 420 (e.g., locking tab) the contents of ampule 415 are dispensed via the hub 400 into the microcatheter 100. The contents of the embolic solution 120 stored in the ampule 405 may be dispensed either simultaneously (i.e., in tandem or at the same time) or non-simultaneously (i.e., sequentially, independently or not at the same time) while subject to aspiration. This simplified hub configuration 400 in FIG. 3 provides control only in the timing associated with the removal, disruption or breaking of the seal 410 on the ampule 405 and dispensing of the embolic solution 120 stored therein. Once the seal 410 is removed, disrupted or broken a controlled portion of the predefined volume of premixed embolic solution 120 may be dispensed via the hub 400 into the microcatheter 100. For example, control of administration of a portion of the total volume of stored contents in the ampule may be realized in preset volume increments (e.g., for each 360 degree rotation of a threaded piston a present volume increment of the total volume is delivered) or any desired increment (e.g., depressing the plunger to dispensed a desired portion of the total volume of stored contents in the ampule).

Microcatheter 100 connected to the distal end of hub 400 may represent any of the exemplary configurations set forth in the illustrated examples herein and described above.

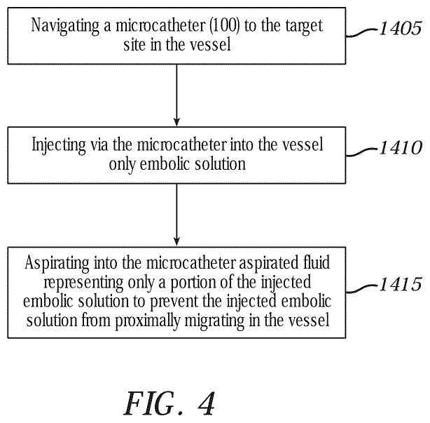

FIG. 4 is an exemplary flow chart of the method of operation of the endovascular embolization system in accordance with the present disclosure wherein aspiration is used to prevent proximal migration of the injected embolic solution. In step 1405 the microcatheter 100 is navigated to the target site in the vessel. With the microcatheter 100 properly positioned at the target site in the vessel 300, in step 1410 the embolic solution 120 is injected via the microcatheter 100 into the vessel 300 at the target site. To prevent proximal migration in the vessel 300 of the injected embolic solution 120, in step 1415 a vacuum pressure is generated via the vacuum source 250 aspirating a portion (i.e., excess) of the injected embolic solution 120 thereby counterbalancing back pressure build-up of the injected embolic solution 120. Furthermore, aspiration of some (e.g., excess) of the injected embolic solution 120 also prevents adherence of the embolic solution to the exterior surface of the outer wall 100c of the microcatheter 100. Still further, aspiration of some (e.g., excess) of the injected embolic solution 120 prevents unintended occlusion of vessels located proximal relative to the target site. Aspiration may be applied either simultaneously (i.e., in tandem or at the same time) or non-simultaneously (i.e., independently, sequentially or not at the same time) as the injection of the embolic solution 120.

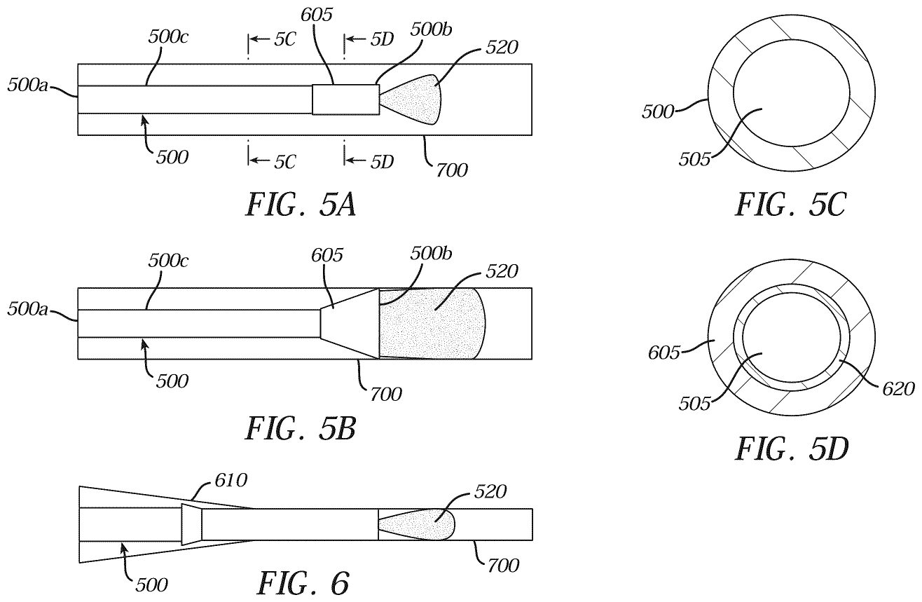

Referring now to FIGS. 5A-10, the present disclosure generally relates to an endovascular embolization treatment by injecting a liquid embolic agent (e.g., liquid glue material) into the vasculature occluding or blocking the supply of blood flow to a target site (e.g., hematoma) experiencing subdural bleeding. Various example embolization treatment systems described and illustrated herein in accordance with the present disclosure by injecting a liquid embolic solution (e.g., an embolic agent such as n-butyl-cyanoacrylate (n-BCA) and oil in any desired ratio) into the vasculature occluding or blocking the supply of blood flow to a target site (e.g., hematoma) experiencing subdural bleeding. Despite the advantages associated with such endovascular embolization treatment, one significant drawback is back pressure from build-up of the injected embolic solution resulting in undesirable migration of the injected embolic solution within the vessel in a proximal direction relative to the location within the vessel where the embolic solution was administered via the microcatheter. Several concerns arise from the proximal migration in the vessel of the injected embolic solution. Vessel(s) downstream in the vasculature relative to the target site of the injected embolic solution may unintentionally and undesirably become occluded from proximal migration of the injected embolic solution. In addition, the injected embolic solution that migrates proximally adheres to the exterior surface of the microcatheter hampering or preventing withdraw from the body. Proximal migration of the injected embolic solution in the vessel is addressed by the present endovascular embolization system and method of treatment by the distal end/tip of the microcatheter itself occluding or blocking the vessel. Occluding or blocking of the vessel may be accomplished using a self-actuating radially expanding occluding component associated disposed at a distal end of the microcatheter. Alternatively, the advancement in the distal direction of the microcatheter to a position wedged (i.e., occluded or blocked) in the vessel may be aided via a mechanical device, lubricious coating and/or ultrasonic vibration.

Several illustrative example configurations of an endovascular embolization system delivering the injected embolic solution into the vessel using a single lumen microcatheter 100 are disclosed herein. Despite being illustrated as having only a single lumen, it is possible and within the scope of the present disclosure to employ a multi-lumen microcatheter.

In the example of FIGS. 5A-5C, the microcatheter 500 has a proximal end 500a and a self-actuating radially expandable occluding member 605 (like an umbrella) at an opposite distal end 500b with an outer wall 500c extending therebetween. A lumen 505 is defined therethrough the microcatheter 500 between the respective ends 500a, 500b (FIG. 5C). Liquid embolic solution 520 (e.g., a solution of an embolic agent (such as n-butyl cyanoacrylate (nBCA)) and oil) is delivered through the lumen 505 of the microcatheter 500 into the vessel 700. The radially expandable occluding member 605 is self-actuating in response to back pressure from blood and/or the injection of embolic solution 520 into the vessel 700 transitioning from a radially constricted state to a radially enlarged state. Preferably, while the radially expandable occluding member 605 is in the radially constricted state, the microcatheter 500 has a substantially uniform outer diameter from its proximal end 500a to its opposite distal end 500b. In the radially expanded state (FIG. 5B) the radially expandable occluding member 605 has a funnel shape or outer contour with a maximum outer diameter along a distal free edge. While in the radially expanded state, the maximum outer diameter of the distal free edge of the radially expandable occluding member 605 is sized to impose sufficient direct physical force against the inner wall of the vessel 700 to prohibit passage in a proximal direction of the injected embolic solution 520. Thus, the distal free edge of the radially expandable occluding member 605 occludes the vessel 700 preventing migration of the injected embolic solution 520 and adherence of the injected embolic solution 520 to the exterior surface of the outer wall 500c of the microcatheter 500 (including the exterior surface of the outer wall of the radially expandable occluding member 605). Adherence along an interior surface of the radially expandable occluding member 605 of the captured or collected injected embolic solution 520 may prohibit subsequent withdrawal of the microcatheter 500. To permit subsequent withdrawal of the microcatheter 500, the radially expandable occluding member 605 is detachable (e.g., electrolytically severable or mechanically releasable) from the main shaft section of the microcatheter 500 disposed proximally thereof. In addition, or alternatively, along an interior surface the radially expandable occluding member 605 may include a non-stick coating or layer 620 (e.g., polytetrafluoroethylene (PTFE)) (FIG. 5D) to prevent or minimize adherence of the collected or captured injected embolic solution 520 allowing the same microcatheter to deliver the embolic solution 520 to multiple distinct locations in the vasculature. Injection of a glucose solution (e.g., dextrose solution) is preferably used to assist in clearing the previously injected liquid embolic solution 520 from the inner diameter of the lumen of the microcatheter. In the illustrated example of FIGS. 5A-5D microcatheter 500 has a single lumen 505 defined therein, but alternatively, a dual lumen microcatheter 500 may be employed with the lumen arranged either concentrically or eccentrically.

Another example microcatheter for use in the endovascular embolization system in accordance with the present disclosure is depicted in FIG. 6 includes a cylindrical tube microcatheter having a sail, fin or parachute 610 disposed radially, preferably 360 degrees, about an exterior surface of the outer wall 500c of the microcatheter 500. In a longitudinal or axial direction, sail 610 is preferably disposed between a proximal end and a midsection point (i.e., locating approximately midway between the proximal and distal ends 500a, 500b, respectively) of the microcatheter 500. Distally of the sail 610 a distal section of the microcatheter 500 including the distal end/tip is uniform and constant (i.e., non-changing or not radially expandable) in the longitudinal or axial direction. Sail 610 has a free proximal edge with a maximum outer diameter and an opposite distal edge secured to the exterior surface of the outer wall 500c of the microcatheter 500. Blood flow captured beneath the free proximal edge of the sail 610 aids in advancement of (i.e., pushing or propelling) the microcatheter 500 distally into the vessel 700 until eventually the distal tip/end 500b of the microcatheter 500 becomes wedged, lodged, occluded or blocked within the vessel 700. In the vessel 700, at the location where the distal end or tip 500b of the microcatheter 500 is wedged the subsequently distally injected embolic solution 520 is prevented from passing in the proximal direction and thus adhering to the exterior surface of the outer wall 500c of the microcatheter 500. Furthermore, the back pressure built-up from the injected embolic solution 520 is prevented from migrating in a proximal direction beyond the distal end 500b of the microcatheter 500 wedged in position in the vessel 700. In addition, vessel(s) disposed proximally of the wedged position are prevented from unintentionally becoming occluded by the injected embolic solution 520 migrating in a proximal direction.

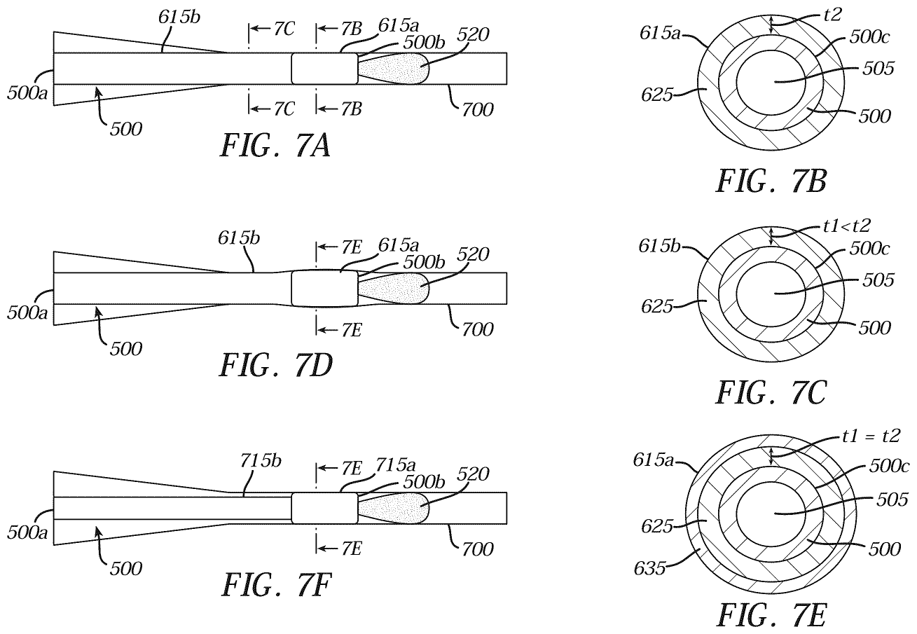

Distal advancement of the microcatheter may be realized by the microcatheter 500 having a non-uniform coefficient of friction in a longitudinal/axial direction. Specifically, a distal section 615b (including the distal end/tip 500b) of the microcatheter 500 having a first coefficient of friction and a main shaft section 615a of the microcatheter 500 disposed proximally relative to the distal section 615b having a second coefficient of friction, as illustrated in the examples shown in FIGS. 7A-7F. The first coefficient of friction of the distal section 615b is selected to be lower than the second coefficient of friction of the main shaft section 615a.

In the example in FIG. 7A, the distal section 615a has a first outer diameter that is substantially equal to a second outer diameter of the main shaft section 615b. The first outer diameter of the distal section 615a is substantially equal to the inner diameter of the vessel 700 without radially enlarging the vessel. The lower coefficient of friction of the distal section 615b fosters advancement distally of the distal section 615a of the microcatheter 500 to a position wedged in thereby occluding (i.e., blocking) the vessel 700 preventing proximal migration of the injected embolic solution 520.

In the alternative examples of FIGS. 7D & 7E the first outer diameter of the distal section 615a is larger than the second outer diameter of the main shaft section 615b. Specifically, FIG. 7D is a first example wherein the first outer diameter of the distal section 615a is greater than the inner diameter of the vessel 700 in which it is being advanced thereby increasing (radially expanding) in size the wall of the vessel 700 to accommodate therein the first outer diameter OD₁ of the distal section 615b of the microcatheter 500 when wedged therein. Accordingly, such tight fit (enhanced wedged effect) ensures that proximal migration and adherence to the external surface of the outer wall of the microcatheter 500 of the subsequently injected embolic solution 520 is prevented. FIG. 7F depicts the first outer diameter of the distal section 615a being less than or equal to the inner diameter of the vessel 700 in which it is being advanced. The first outer diameter of the distal section 615a of the microcatheter assisted by the lower coefficient of friction is wedged within the vessel 700 without radially expanding the wall of the vessel 700.

Different ways are recognized to attain a non-uniform coefficient of friction in the longitudinal/axial direction (e.g., different friction coefficients for the distal and main shaft sections 615a, 615b, respectively) of the microcatheter 500. Typically, microcatheters are manufactured to include a hydrophilic outer layer or coating 625 to provide a slippery surface to assist during navigation to the target site in the vessel 700. A non-uniform radial thickness of a single material hydrophilic coating or layer 625 may be applied to the microcatheter 500 so that the distal section 615a has a first radial thickness t1 (FIG. 7B) greater than a second radial thickness t2 of the main shaft section 615b (FIG. 7C), wherein t1>t2. For example, the first radial thickness t1 in the distal section 615a may be in the range of approximately 30 microns-approximately 300 microns, while the second radial thickness t2 in the main shaft section 615b may be in the range of approximately 5 microns to approximately 50 microns. Otherwise, the non-uniform coefficient of friction in the longitudinal/axial direction may be achieved by applying a supplemental lubricant (e.g., silicone based lubricant 635) over the hydrophilic outer layer or coating 625 along the distal section 615a. A single hydrophilic outer layer or coating 625 of uniform radial thickness in the longitudinal/axial direction is applied to the entire microcatheter (including the distal section 615a and the main shaft section 615b). A supplemental lubricant (e.g., silicone lubricant) coating 635 is applied over the hydrophilic coating 625 in the distal section 615a. Therefore, the distal section 615a with the supplemental lubricant coating 635 has a lower coefficient of friction relative to the higher coefficient of friction of the main shaft 615b having only the hydrophilic outer layer or coating 625 (free of the supplemental lubricant coating 635). In addition, along at least portion of an interior surface the lumen of the microcatheter may include a non-stick coating or layer (e.g., hydrophilic or oil) (similar to that depicted in FIG. 5D) to prevent or minimize adherence of the collected or captured injected embolic solution 520 allowing the same microcatheter to deliver the embolic solution 520 to multiple distinct locations in the vasculature. Injection of a glucose solution (e.g., dextrose solution) is preferably used to assist in clearing the previously injected liquid embolic solution 520 from the inner diameter of the lumen of the microcatheter. Alternatively, the distal section 615a and the main shaft 615b may have a substantially equal coefficient of friction. Regardless of the coefficient of friction of each of the distal section 615a and the main shaft 615b, with the absorption of a liquid (e.g., water) the hydrophilic coating self-adjusts swelling radially outward (i.e., increasing in radial thickness). Radial hydrated thickness refers to the radial thickness of the hydrophilic coating when in a radially swelled state following absorption of the liquid, while radial non-hydrated thickness refers to the radial thickness of the hydrophilic coating prior to absorption of the liquid. By way of example, the main shaft 615b has a radial hydrated thickness in a range of approximately 10 microns to approximately 50 microns, while the associated distal shaft 615a has a radial hydrated thickness in a range of approximately 30 microns to approximately 300 microns. For this example, the corresponding radial non-hydrated thickness of the main shaft 615b is in the range of approximately 1 micron to approximately 10 microns, while the associated radial non-hydrated thickness of the distal shaft 615a is in a range of approximately 10 microns to approximately 50 microns.

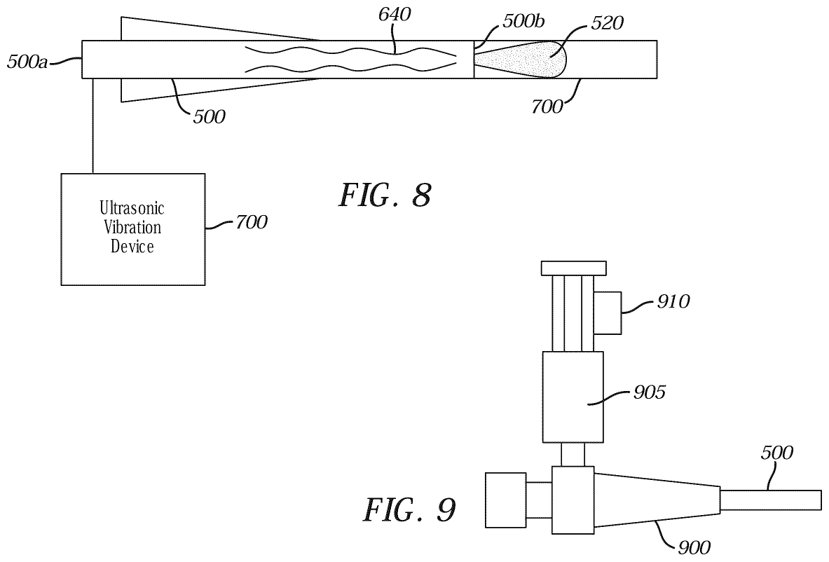

Still further it is contemplated to employ ultrasonic vibration to aid or assist in distal advancement of the microcatheter 500 to a wedged position in direct physical contact with the wall of the vessel 700 occluding (i.e., blocking) proximal migration and adherence to the exterior surface of the outer wall 500c of the microcatheter 500 of the injected embolic solution 520. An ultrasonic source 800 is electrically connected to apply ultrasonic vibrations 805 to the distal end/tip 500b of the microcatheter 500, as shown in FIG. 8. The ultrasonic vibrations 805 aid or assist in distal advancement of the microcatheter 500 through the vessel 700 by reducing friction lockup (i.e., micro surface contact release of the exterior surface of the microcatheter 500 and internal wall of the vessel 700). Moreover, the applied ultrasonic vibrations may also relax the wall of the vessel 700.

FIG. 9 is an example dual channel hub 900 in which an ampule 905 preloaded with a predefined volume of the embolic solution 520 (e.g., premixture in a desired ratio of an embolic agent and oil) is attached or fitted onto a first lure connector of hub 900. Ampule 905 is sealed thereby preventing premature dispensing of the contents stored therein. For example, ampule 905 may include a corresponding locking tab 910 that when removed, disrupted or broken by the physician or interventionalist dispenses the preloaded premixed solution stored therein. In response to removing, disrupting or breaking a seal 920 (e.g., locking tab) the contents of ampule 915 are dispensed via the hub 900 into the microcatheter 500. This simplified hub configuration 900 in FIG. 9 provides control only in the timing associated with the removal, disruption or breaking of the seal 910 on the ampule 905 and dispensing of the embolic solution 520 stored therein. Once the seal 910 is removed, disrupted or broken the entire predefined volume of premixed embolic solution 520 is dispensed via the hub 900 into the microcatheter 500.

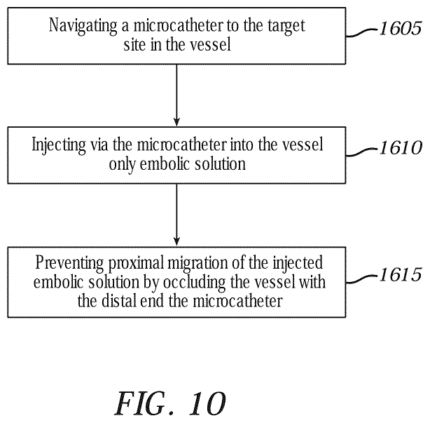

FIG. 10 is an exemplary flow chart of the method of operation of the endovascular embolization system in accordance with the present disclosure wherein aspiration is used to prevent proximal migration of the injected embolic solution. In step 1605 the microcatheter 500 is navigated to the target site in the vessel. With the microcatheter 500 properly positioned at the target site in the vessel 700, in step 1610 the embolic solution 520 is injected via the microcatheter 500 into the vessel 700 at the target site. To prevent proximal migration in the vessel 700 of the injected embolic solution 520, in step 1615 the vessel 700 is occluded (i.e., blocked) by the distal end 500b the microcatheter 500. Various method for occluding the vessel with the distal end of the microcatheter are presented herein. One way to occlude the vessel is via a self-actuating radially expandable occluding member 605 representing the distal end 500b of the microcatheter 500 and transitioning to the radially enlarged state in response to back pressure from blood and/or the injection of the embolic solution 520 into the vessel 700. Optionally, the self-actuating radially expandable occluding member 605 may be detachable to allow withdraw of the main shaft section of the microcatheter 500 while the self-actuating radially expandable occluding member 605 remains in place within the vessel. It is also possible to coat the interior surface of the self-actuating radially expandable occluding member 605 with a non-stick coating or layer 620 to prevent adherence of the injected embolic solution 520 captured or collected therein.

Otherwise, the distal end 500b of the microcatheter may be non-radially expandable, wherein the vessel 700 is occluded by aiding distal advancement in the vessel 700 of the microcatheter 500 so that the non-radially expandable distal end 500b is in a wedged position in direct physical contact with a wall of the vessel 700. Distal advancement of the distal end of the microcatheter to a wedged position in the vessel may be assisted via a self-actuating distal advancing member 610 (e.g., sail, parachute or fin), applying to the outer surface 500c of the microcatheter 500 having a non-uniform coefficient of friction in a longitudinal/axial direction and/or applying ultrasonic vibrations to the distal end/tip of the microcatheter 500.

Aspects of the present disclosure are also provided by the following numbered clauses:
Clause 1: A method for embolization treatment at a target site within a vessel (300) using an endovascular embolization system, the method comprising the steps of: navigating a microcatheter (100) to the target site in the vessel (300); and injecting via the microcatheter (100) into the vessel (300) only embolic solution (120); and aspirating into the microcatheter (100) aspirated fluid representing only a portion of the injected embolic solution (120) to prevent the injected embolic solution (120) from proximally migrating in the vessel (300).
Clause 2: The method of Clause 1, wherein the aspiration of only the portion of the injected embolic solution (120) in the vessel (300) prevents adhering of the injected embolic solution (120) to an exterior surface of the microcatheter (100).
Clause 3: The method of any of Clauses 1 through 2, wherein the aspiration of only the portion of the injected embolic solution (120) in the vessel prevents unintentional occluding of the vessel (300) proximally of the target site of the injected embolic solution (120).
Clause 4: The method of any of Clauses 1 through 3, wherein the microcatheter (100) has a proximal end (100a), an opposite distal end (100b) and an outer sidewall (100c) extending longitudinally between the proximal end (100a) and the distal end (100b) defining a first lumen (105) receiving the embolic solution (120); the microcatheter further including a second lumen (115) through which only the portion of the injected embolic solution (120) in the vessel (300) is aspirated, the second lumen (115) being separate from the first lumen (105); wherein the first lumen (105) has a first inlet port (105a) and a first outlet port (105b), while the second lumen (115) has a second inlet port (115a) receiving only the portion of the injected embolic solution (120) being aspirated and an opposite second outlet port (115b).
Clause 5: The method of Clause 4, wherein the second lumen (115) is arranged radially outward relative to the first lumen (105).
Clause 6: The method of Clause 5, wherein the first lumen (105) and the second lumen (115) are arranged concentrically of one another.
Clause 7: The method of Clause 5, wherein the first lumen (105) and the second lumen (115) are arranged eccentrically relative to one another.
Clause 8: The method of Clause 4, wherein the second inlet port (115a) of the second lumen (115) is disposed in a longitudinal direction distally of the first outlet port (105b) of the first lumen (105) thereby aspirating blood distally of the target site of the injection into the vessel of the embolic solution (120) creating distally of the first outlet port (105b) a suction of negative pressure drawing distally through the vessel (300) towards the target site the injected embolic solution (120).
Clause 9: The method of Clause 4, wherein the second inlet port (115a) of the second lumen (115) is aligned in a longitudinal direction with the second outlet port (105b) of the first lumen (105).
Clause 10: The method of any of Clauses 1 through 9, wherein the aspirating step occurs simultaneously with the step of injecting only the embolic solution (120) into the vessel (300).
Clause 11: The method of any of Clauses 1 through 9, wherein the aspirating step occurring independently of and prior to the step of injecting only the embolic solution (120) into the vessel (300) aspirates blood from the vessel (300) prior to the injection of only the embolic solution (120) into the vessel (300).
Clause 12: The method of any of Clauses 1 through 11, wherein the embolic solution (120) includes n-butyl cyanoacrylate.
Clause 13: The method of Clause 1, wherein the embolic solution (120) comprises an embolic agent and an oil premixed and preloaded in an ampule (405) having a seal (410) wherein the ampule (405) is fluidly connected to an administering device (400) and includes a seal (410) preventing premature dispensing of the embolic solution (120) contained in the ampule (405); and the injecting step comprises, in response to disrupting the seal (410), dispensing the embolic solution (120) from the ampule (405) into the microcatheter (100) via the administering device (400) interconnected therebetween.
Clause 14: The method of any of Clauses 1 through 13, wherein the aspirated fluid includes blood.
Clause 15: An endovascular embolization system comprising: a microcatheter (100) having a proximal end (100a), an opposite distal end (100b) and an outer sidewall (100c) extending longitudinally between the proximal end (100a) and the distal end (100b) defining a first lumen (105) through which only an embolic solution (120) is injectable; and separate from the first lumen (105), the microcatheter (100) further including a second lumen (115) into which aspirated fluid representing only a portion of the embolic solution (120) once injected from the microcatheter (100) is receivable preventing the once injected embolic solution (120) from proximally migrating; wherein the first lumen (105) has a first inlet port (105a) and a first outlet port (105b), while the second lumen (115) has a second inlet port (115a) receiving only the portion of the injected embolic solution (120) being aspirated and an opposite second outlet port (115b).
Clause 16: The system of Clause 15, wherein the aspiration of only the portion of the once injected embolic solution (120) prevents adhering of the once injected embolic solution (120) to an exterior surface of the microcatheter (100).
Clause 17: The system of Clause 15, wherein the second lumen (115) is arranged radially outward relative to the first lumen (105).
Clause 18: The system of Clause 15, wherein the first lumen (105) and the second lumen (115) are arranged concentrically of one another.
Clause 19: The system of Clause 15, wherein the first lumen (105) and the second lumen (115) are arranged eccentrically relative to one another.
Clause 20: The system of Clause 15 wherein the second inlet port (115a) of the second lumen (115) is disposed in a longitudinal direction distally of the first outlet port (105b) of the first lumen (105) thereby aspirating blood distally of the target site of the injection into the vessel of the embolic solution (120) creating distally of the first outlet port (105b) a suction of negative pressure drawing distally through the vessel (300) towards the target site the injected embolic solution (120).
Clause 21: The system of Clause 15, wherein the second inlet port (115a) of the second lumen (115) is aligned in a longitudinal direction with the second outlet port (105b) of the first lumen (105).
Clause 22: The system of Clause 15, wherein the embolic solution (120) is injectable via the first lumen (105) simultaneously with the aspirated fluid receivable in the second lumen (115).
Clause 23: The system of Clause 15, wherein the embolic solution (120) is injectable via the first lumen (105) independently of and prior to the aspirated fluid receivable in the second lumen (115).
Clause 24: The system of Clause 15, wherein the embolic solution (120) includes n-butyl cyanoacrylate.
Clause 25: The system of Clause 15, wherein the embolic solution (120) comprises an embolic agent and an oil premixed and preloaded in an ampule (405) having a seal (410) wherein the ampule (405) is fluidly connected to an administering device (400) and includes a seal (410) preventing premature dispensing of the embolic solution (120) contained in the ampule (405).
Clause 26: The system of Clause 15, wherein the aspirated fluid includes blood.
Clause 27: A method for embolization treatment at a target site within a vessel (700) using an endovascular embolization system, the method comprising the steps of: navigating a microcatheter (500) to the target site in the vessel (700); the microcatheter (500) having a proximal end (500a), an opposite distal end (500b) and an outer surface (500c) extending therebetween; injecting via the microcatheter (500) into the vessel (700) only embolic solution (520); and preventing proximal migration of the injected embolic solution (520) by occluding the vessel (700) with the distal end (500b) of the microcatheter (500).
Clause 28: The method of Clause 27, wherein the vessel (700) is occluded via a self-actuating radially expandable occluding member (605) representing the distal end (500b) of the microcatheter (500).
Clause 29: The method of Clause 27, wherein the distal end (500b) of the microcatheter is non-radially expandable and the vessel (700) is occluded by aiding distal advancement in the vessel (700) of the microcatheter (500) so that the non-radially expandable distal end (500b) is in a wedged position in direct physical contact with a wall of the vessel (700).
Clause 30: The method of Clause 28, wherein the step of occluding the vessel (700) comprises transitioning of the self-actuating radially expandable occluding member (605) disposed about the microcatheter (500) to a radially enlarged state.
Clause 31: The method of Clause 30, wherein the radially self-actuating radially expandable occluding member (605) is a tapered funnel having a maximum outer diameter at a distal free edge.
Clause 32: The method of any of Clauses 30 through 31, wherein the self-actuating radially expandable occluding member (605) transitions to the radially enlarged state in response to back pressure from blood and/or the injection of the embolic solution (520) into the vessel (700).
Clause 33: The method of any of Clauses 30 through 32, wherein after transitioning of the self-actuating radially expandable occluding member (605) to the radially enlarged state, further comprising detaching the self-actuating radially expandable occluding member (605) from the microcatheter (500); and subsequently withdrawing the microcatheter (500) from the vessel (700) while the detached radially self-expanding occluding member (605) remains adhered in place in the vessel (700) via the injected embolic solution (520).
Clause 34: The method of any of Clauses 30 through 33 wherein the self-actuating radially expanding occluding component (605) has a non-stick coating (620) along an interior surface preventing adherence thereto of the injected embolic solution (520) collected therein.
Clause 35: The method of Clause 29, wherein the aiding in distal advancement of the microcatheter (500) to the wedged position in the vessel (700) is via a self-actuating distal advancing member (610) secured about the outer surface (500c) of the microcatheter (500) and having a free proximal edge.
Clause 36: The method of Clause 35, wherein, the aiding in distal advancement of the microcatheter (500) to the wedged position in the vessel (700) is via blood flow and/or blood pressure imparting a force on the self-actuating distal advancing member (610).
Clause 37: The method of Clause 29 wherein the aiding in distal advancement of the microcatheter to the wedged position in the vessel (700) comprises the outer surface (500c) of the microcatheter (500) having a non-uniform coefficient of friction in a longitudinal direction.
Clause 38: The method of Clause 37, wherein the outer surface (500c) of the microcatheter includes a distal section (615a) having a first outer diameter and a first coefficient of friction and a main shaft section (615b) disposed proximally of the distal section (615a) having a second outer diameter and a second coefficient of friction less than the first coefficient of friction of the distal section (615a).
Clause 39: The method of Clause 38, wherein the first outer diameter of the distal section (615a) is greater than the second outer diameter of the main shaft section (615b).
Clause 40: The method of Clause 38, wherein the microcatheter (500) has a hydrophilic coating (625) of non-uniform radial thickness in a longitudinal direction, the hydrophilic coating (625) having a first radial thickness (t1) in the distal section (615a) a second thickness (t2) in the main shaft section (615b), wherein the first radial thickness (t1) is greater than the second radial thickness (t2).
Clause 41: The method of Clause 38, wherein the microcatheter (500) has a hydrophilic coating (625) of non-uniform radial thickness in a longitudinal direction, the hydrophilic coating (625) having a first radial hydrated thickness (t1) in the distal section (615a) a second radial hydrated thickness (t2) in the main shaft section (615b), wherein the first radial hydrated thickness (t1) is greater than the second radial hydrated thickness (t2).
Clause 42: The method of Clause 38, wherein the microcatheter (500) is covered with a hydrophilic coating (625) of substantially uniform radial thickness in a longitudinal direction in the respective distal and main shaft section (615a, 615b); and the distal section (615a) further comprises a supplemental lubricant (630) applied over the hydrophilic coating (625).
Clause 43: The method of Clause 38, wherein the supplemental lubricant (635) is a silicone base lubricant.
Clause 44: The method of Clause 29, wherein the aiding in distal advancement of the microcatheter (500) to the wedged position in the vessel (700) comprises applying ultrasonic vibrations (640) generated by an ultrasonic vibrating device (800) to the microcatheter (500) causing discrete regions of release in contact surface between the outer surface (500c) of the microcatheter (500) and wall of the vessel (700).
Clause 45: The method of Clause 44, wherein the applied ultrasonic vibrations (640) relax the vessel (700).
Clause 46: The method of any of Clauses 27 through 45, wherein the embolic solution is n-butyl cyanoacrylate.
Clause 47: The method of Clause 27, wherein the embolic solution (520) comprises an embolic agent and an oil premixed and preloaded in an ampule (905) having a seal (910); and the injecting step comprises, in response to disrupting the seal (910), dispensing the embolic solution (520) from the ampule (905) into the microcatheter (500) via a hub (900) interconnected therebetween.
Clause 48: An endovascular embolization system comprising: a microcatheter (500) having a proximal end (500a), an opposite distal end (500b) and an outer surface (500c) extending therebetween; wherein the microcatheter (500) has a passageway defined therein through which only embolic solution (520) is injectable; and a member aiding distal advancement of the microcatheter (500) preventing proximal migration of the embolic solution (520) once injected from the microcatheter (500).
Clause 49: The system of Clause 48, wherein the member aiding distal advancement is a self-actuating radially expandable occluding member (605) representing the distal end (500b) of the microcatheter (500).
Clause 50: The system of Clause 48, wherein the distal end (500b) of the microcatheter is non-radially expandable.
Clause 51: The system of Clause 49, wherein the self-actuating radially expandable occluding member (605) disposed about the microcatheter (500) is transitionable from a radially compressed state to a radially enlarged state.
Clause 52: The system of Clause 51, wherein the self-actuating radially expandable occluding member (605) is a tapered funnel having a maximum outer diameter at a distal free edge.
Clause 53: The system of any of Clauses 51 through 52, wherein the self-actuating radially expandable occluding member (605) transitions to the radially enlarged state in response to back pressure from blood and/or the embolic solution (520) once injected from the microcatheter (500).
Clause 54: The system of any of Clauses 51 through 53, wherein the self-actuating radially expandable occluding member (605) while in the radially enlarged state is detachable from the microcatheter (500).
Clause 55: The system of any of Clauses 31 through 54, wherein the self-actuating radially expanding occluding component (605) has a non-stick coating (620) along an interior surface preventing adherence thereto of the embolic solution (520).
Clause 56: The system of Clause 50, wherein the member aiding in distal advancement member is self-actuating, secured about the outer surface (500c) of the microcatheter (500) and having a free proximal edge.
Clause 57: The system of Clause 56, wherein the member aiding in distal advancement is self-actuated via blood flow and/or blood pressure.
Clause 58: The system of Clause 50, wherein the member aiding in distal advancement comprises the outer surface (500c) of the microcatheter (500) having a non-uniform coefficient of friction in a longitudinal direction.
Clause 59: The system of Clause 58, wherein the outer surface (500c) of the microcatheter includes a distal section (615a) having a first outer diameter and a first coefficient of friction and a main shaft section (615b) disposed proximally of the distal section (615a) having a second outer diameter and a second coefficient of friction less than the first coefficient of friction of the distal section (615a).
Clause 60: The system of Clause 59, wherein the first outer diameter of the distal section (615a) is greater than the second outer diameter of the main shaft section (615b).
Clause 61: The system of Clause 59, wherein the microcatheter (500) has a hydrophilic coating (625) of non-uniform radial thickness in a longitudinal direction, the hydrophilic coating (625) having a first radial thickness (t1) in the distal section (615a) a second thickness (t2) in the main shaft section (615b), wherein the first radial thickness (t1) is greater than the second radial thickness (t2).
Clause 62: The system of Clause 59, wherein the microcatheter (500) has a hydrophilic coating (625) of non-uniform radial thickness in a longitudinal direction, the hydrophilic coating (625) having a first radial hydrated thickness (t1) in the distal section (615a) a second radial hydrated thickness (t2) in the main shaft section (615b), wherein the first radial hydrated thickness (t1) is greater than the second radial hydrated thickness (t2).
Clause 63: The system of Clause 59, wherein the microcatheter (500) is covered with a hydrophilic coating (625) of substantially uniform radial thickness in a longitudinal direction in the respective distal and main shaft section (615a, 615b); and the distal section (615a) further comprises a supplemental lubricant (630) applied over the hydrophilic coating (625).
Clause 64: The system of Clause 63, wherein the supplemental lubricant (635) is a silicone bae lubricant.
Clause 65: The system of Clause 50, wherein the member aiding in distal advancement comprises an ultrasonic vibrating device (800) applying ultrasonic vibrations (640) to the microcatheter (500) causing discrete regions of release in contact surface between the outer surface (500c) of the microcatheter (500) and wall of the vessel (700).
Clause 66: The system of any of Clauses 48 through 65, wherein the embolic solution is n-butyl cyanoacrylate.
Clause 67: The system of Clause 48, further comprising an ampule (905) having a seal (910) and preloaded with the embolic solution (520) comprising premixed embolic agent and oil; and the seal (910) being disruptable dispensing therefrom the embolic solution (520) from the ampule (905) into the microcatheter (500) via a hub (900) interconnected therebetween.

The descriptions contained herein are examples of embodiments of the invention and are not intended in any way to limit the scope of the invention. As described herein, the invention contemplates many variations and modifications of a method for embolization treatment at a target site within a vessel using an endovascular embolization system wherein proximal migration in the vessel of injected embolic solution is prevented by aspirating some (i.e., excess) of the injected embolic solution. Modifications and variations apparent to those having skilled in the pertinent art according to the teachings of this disclosure are intended to be within the scope of the claims which follow.

What is claimed is:

1. A method for embolization treatment at a target site within a vessel using an endovascular
embolization system, the method comprising the steps of:
navigating a microcatheter to the target site in the vessel; and
injecting via the microcatheter into the vessel only embolic solution; and
aspirating into the microcatheter aspirated fluid representing only a portion of the injected embolic solution to prevent the injected embolic solution from proximally migrating in the vessel.

2. The method of claim 1, wherein the aspiration of only the portion of the injected embolic solution in the vessel prevents adhering of the injected embolic solution to an exterior surface of the microcatheter.

3. The method of claim 1, wherein the aspiration of only the portion of the injected embolic solution (120) in the vessel prevents unintentional occluding of the vessel proximally of the target site of the injected embolic solution.

4. The method of claim 1, wherein the microcatheter has a proximal end, an opposite distal end and an outer sidewall extending longitudinally between the proximal end and the distal end defining a first lumen receiving the embolic solution; the microcatheter further including a second lumen through which only the portion of the injected embolic solution in the vessel is aspirated, the second lumen being separate from the first lumen; wherein the first lumen has a first inlet port and a first outlet port, while the second lumen has a second inlet port receiving only the portion of the injected embolic solution being aspirated and an opposite second outlet port.

5. The method of claim 4, wherein the second lumen is arranged radially outward relative to the first lumen.

6. The method of claim 5, wherein the first lumen and the second lumen are arranged concentrically of one another.

7. The method of claim 5, wherein the first lumen and the second lumen are arranged eccentrically relative to one another.

8. The method of claim 4, wherein the second inlet port of the second lumen is disposed in a longitudinal direction distally of the first outlet port of the first lumen thereby aspirating blood distally of the target site of the injection into the vessel of the embolic solution creating distally of the first outlet port a suction of negative pressure drawing distally through the vessel towards the target site the injected embolic solution.

9. The method of claim 4, wherein the second inlet port of the second lumen is aligned in a longitudinal direction with the second outlet port of the first lumen.

10. The method of claim 1, wherein the aspirating step occurs simultaneously with the step of injecting only the embolic solution into the vessel.

11. A method for embolization treatment at a target site within a vessel using an endovascular embolization system, the method comprising the steps of:
navigating a microcatheter to the target site in the vessel; the microcatheter having a proximal end, an opposite distal end and an outer surface extending therebetween;
injecting via the microcatheter into the vessel only embolic solution; and
preventing proximal migration of the injected embolic solution by occluding the vessel with the distal end of the microcatheter.

12. The method of claim 11 wherein the vessel is occluded via a self-actuating radially expandable occluding member representing the distal end of the microcatheter.

13. The method of claim 11, wherein the distal end of the microcatheter is non-radially expandable and the vessel is occluded by aiding distal advancement in the vessel of the microcatheter so that the non-radially expandable distal end is in a wedged position in direct physical contact with a wall of the vessel.

14. The method of claim 12, wherein the step of occluding the vessel comprises transitioning of the self-actuating radially expandable occluding member disposed about the microcatheter to a radially enlarged state.

15. The method of claim 14, wherein the radially self-actuating radially expandable occluding member is a tapered funnel having a maximum outer diameter at a distal free edge.

16. The method of claim 15, wherein the self-actuating radially expandable occluding member transitions to the radially enlarged state in response to back pressure from blood and/or the injection of the embolic solution into the vessel.

17. The method of claim 16, wherein after transitioning of the self-actuating radially expandable occluding member to the radially enlarged state, further comprising detaching the self-actuating radially expandable occluding member from the microcatheter; and subsequently withdrawing the microcatheter from the vessel while the detached radially self-expanding occluding member remains adhered in place in the vessel via the injected embolic solution.

18. The method of claim 17, wherein the self-actuating radially expanding occluding component has a non-stick coating along an interior surface preventing adherence thereto of the injected embolic solution collected therein.

19. The method of claim 13, wherein the aiding in distal advancement of the microcatheter to the wedged position in the vessel is via a self-actuating distal advancing member secured about the outer surface of the microcatheter and having a free proximal edge.

20. The method of claim 19, wherein, the aiding in distal advancement of the microcatheter 1 to the wedged position in the vessel 1 is via blood flow and/or blood pressure imparting a force on the self-actuating distal advancing member.
